(19) United States
(12) Patent Application Publication
(10) Pub. No.: US 2021/0210171 A1
(43) Pub. Date: Jul. 8, 2021

(54) A METHOD OF STORING INFORMATION USING DNA MOLECULES

(71) Applicants: VIB VZW, Gent (BE); KATHOLIEKE UNIVERSITEIT LEUVEN, K.U.LEUVEN R&D, Leuven (BE)

(72) Inventors: Rocco Stirparo, Leuven (BE); Jan Cools, Herent (BE); Flora D'Anna, Leuven (BE); Matthieu Moisse, Kessel-Lo (BE); Juan Fernandez Garcia, Leuven (BE); Antonio Ammirati, Kessel-Lo (BE)

(21) Appl. No.: 17/058,454

(22) PCT Filed: Jun. 7, 2019
(86) PCT No.: PCT/EP2019/064928

§ 371 Date: Nov. 24, 2020

(30) Foreign Application Priority Data
Jun. 7, 2018 (EP) 18176614.8

(51) Int. Cl. G16B 50/20; G16B 30/00; G06F 11/10; G06F 3/06

(52) U.S. Cl. CPC G16B 50/20; G16B 30/00; G06F 3/0673; G06F 3/0608; G06F 3/0659; G06F 11/1076

(57) ABSTRACT

A method of storing information using DNA molecules is disclosed. The method comprises converting (100) a file of information into a plurality of fragments, wherein the plurality of fragments comprise a plurality of bytes. This plurality of bytes is converted (110) into a plurality of nucleotides using selected ones of a plurality of dictionaries and a file unit is constructed (120, 130, 140) comprising the plurality of nucleotides and an identification of the used ones of the plurality of dictionaries. Finally, a plurality of DNA molecules is synthesized (150) from the constructed file.

### CROSS REFERENCE TO RELATED APPLICATIONS

This application is a national phase entry under 35 U.S.C. § 371 of International Patent Application PCT/EP2019/064928, filed Jun. 7, 2019, designating the United States of America and published in English as International Patent Publication WO 2019/234213 on Dec. 12, 2019, which claims the benefit under Article 8 of the Patent Cooperation Treaty to European Patent Application Serial No. 18176614.8, filed Jun. 7, 2018, the entireties of which are hereby incorporated by reference.

### FIELD OF THE INVENTION

The invention relates to a method of storing information using DNA molecules. More precisely a novel reverse translation method is disclosed herein.

### BACKGROUND OF THE INVENTION

Data storage needs are growing exponentially and currently doubling every three years. At this speed, in the next 30 years there will be at least 1000 times more information to store. Unfortunately, current technologies for storing information are already consuming too many resources and therefore data storage will soon become unsustainable. There is therefore a need to develop a new storage medium that consumes less resources, occupies less physical space and is stable for very long periods.

DNA is a promising medium for storing data. DNA storage systems require very low maintenance and the DNA molecule remains stable for hundreds of years. The DNA molecule is currently the most compact way of storing information, thus reducing the requirement of physical space. There are however some limitations with current DNA storage systems. For example, homopolymers, repetitions and mis-balance of G/C content are currently incompatible with DNA synthesis and sequencing technologies. DNA sequences should be preferentially random and highly diverse while digital data, which will be encoded in the sequences of the DNA molecules, are often very organized and repetitive. Moreover, synthesis, amplification and sequencing of the DNA molecules may create some mutations, which require redundancy and correction algorithms in order to keep the information accurate.

In the last years, there have been several studies and patent applications that have demonstrated that data storage is possible by using small DNA molecules (oligonucleotides with a length of less than 200 nucleotides) or larger DNA molecules (>200 nucleotides). Digital information has been translated into DNA in a linear way and/or by first randomizing the binary source. Examples of the linear translation method are Church et al. (2012 Science 337:1628) that used a basic algorithm translating every bit 0 into A/C and every bit 1 into T/G and Goldman et al. (2013 Nature 494:77-80) that translated the binary code into trinary code in order to avoid homopolymers. Their international patent applications are respectively No. WO 2014/014991 and WO 2013/178801, and both teach a method of storing information in DNA nucleotides. In these patent applications, oligonucleotides are synthesized. However, these methods have been found to be pretty sensitive to long repetitions and mutations. As a result, this can lead to incomplete recovery of the digital files and thus loss of information.

An alternative approach is to adjust the digital code first in order to obtain easy synthesizable DNA molecules and to anticipate sequencing problems afterwards. For example, Organick et al. (2018 Nat Biotech 36: 242-249) translated 200 megabytes of data into oligonucleotides after randomizing the binary source code. Yadzi et al. (2017 Scientific Reports 7:5011) on the other hand compressed the binary files first in order to reduce the space and to avoid repetitions to some extent. Although optimized formula were used to avoid high G/C content and/or homopolymers, some fragments were still difficult to synthesize and/or sequence.

Other examples of papers discussing storage of information in nucleic acids comprise Zhirnov et al. (2016 Nature Materials 15: 366-370), Ehrlich and Zielinski (2017 Science 355: 950-954) and Tavella et al. (2018, arXiv:1801.04774). Tavella et al. teach a solution which allows digitally encoded information to be stored into non-motile bacteria, which compose an archival architecture of clusters, and to be later retrieved by engineered motile bacteria, whenever reading operations are needed. Tavella et al. used the encoding method described by Goldman with the associated issues mentioned above.

### SUMMARY OF THE INVENTION

All currently available approaches to store digital information into nucleic acids use a forward translation method, i.e. from the digital code to DNA code. However, although DNA synthesis and sequencing technologies have evolved dramatically, not all DNA molecules can be synthesized and/or sequenced with the same efficiency and accuracy. To prevent that DNA molecules comprising homopolymers, repetitions or a misbalance of G/C content should be synthesized, most recent data storage approaches adapt the binary code before translating it. Hence, any in silico translation should still be checked for compatibility with current synthesis and sequencing requirements and adapted if needed.

Here, Applicants disclose a reverse translation approach. The herein described novel data storage methods make use of a set of selected and diverse DNA elements that are optimized for synthesis and sequencing purposes. Each DNA element (which can be seen as a “word”) from said set of DNA elements (which can be seen as a “dictionary”) is then translated into a different byte of digital information. A byte which consists of 8 bits is here mentioned as a non-limiting example. DNA elements can also be translated into stretches of an alternative number of bits, for example 4 bits, 5 bits, 6 bits or 7 bits. Interestingly, the way how a DNA element (or “word”) is translated to (for example) a byte, i.e. the translation key, can be changed. Hence, this approach enables the use of a plurality of dictionaries by simply changing the translation key. The reverse translation methods herein described have several advantages over the prior art methods of storing digital data. First, because of the optimized “words”, any DNA fragment constructed by a combination of said “words” will efficiently be synthesized and sequenced. Second, by changing the translation key (and thus the dictionary used) for every digital element (e.g. a byte) to be translated, even a highly repetitive digital (e.g. binary) code will be converted into a highly diverse and randomized DNA fragment. Third, because any digital data file can be translated into a highly random DNA fragment, long DNA files encoding large digital data fragments can be synthesized. Long DNA fragments can be incorporated in plasmids which are more stable compared to oligonucleotides. Moreover, long DNA fragments significantly increase the information density.

Hence, a novel method is taught in this document to enable the storing of digital data into DNA molecules. The method comprises converting a file of information, representing the digital data, into a plurality of fragments, wherein the plurality of fragments comprises a plurality of binary elements of the digital data. In a next step, the plurality of binary elements is converted into a plurality of nucleotides using selected ones of a plurality of dictionaries and then a file unit is constructed. The file unit comprises the plurality of nucleotides and an identification of the used ones (so called translation key or “mask”, see later) of the plurality of dictionaries. The file unit should further comprise a fragment code indicating the position of the fragment in the file of information as well as a file identifier which corresponds to the number of the file.

The file unit is passed to a synthesizer for synthesizing a plurality of DNA molecules from the constructed file unit, and subsequently the plurality of synthesized DNA molecules is stored. Alternatively phrased, the application provides in a first aspect, a method of storing digital information using DNA molecules, said method comprises the steps of:
converting (100) a file of digital information into a plurality of fragments, wherein the plurality of fragments comprises or can be converted to a plurality of binary elements;
converting (110) the plurality of binary elements into a plurality of nucleotides using selected ones of a plurality of dictionaries;
constructing (120, 130, 140) a file unit comprising the plurality of nucleotides and an identification of the used ones of the plurality of dictionaries;
synthesizing (150) a plurality of DNA molecules from the constructed file unit; and
storing the plurality of synthesized DNA molecules.

The method of this disclosure is able to translate the digital file in both short and long DNA sequences, irrespective of the synthesis limits. The dictionaries used comprise a plurality of members (so-called “words”). In one embodiment, the plurality of members consists of four, five or six nucleotides. In particular embodiments, said members of the dictionaries consisting of five or six nucleotides differ from each other by at least two nucleotides. This improves accuracy of later reading of the DNA sequences by reducing errors due to a mutation in one of the nucleotides. In further embodiments, different ones of the plurality of dictionaries are used for converting (110) ones of the plurality of binary elements.

The DNA molecules are plasmids in one example of the disclosure. The plasmid is a small circular DNA molecule capable of replicating autonomously inside a bacterium. In one aspect two or three different plasmids are synthesized, but this is not limiting of the invention, and stored per fragment of the digital data. In the event that the information in one of the plasmids cannot be decoded, then there is one or two further plasmids which encode the same item of information and from which it should be possible to decode the fragment containing the item of information. In another embodiment, the above methods are provided wherein the file unit further comprises a fragment code indicating position of the fragment in the file of digital information.

In another aspect, collections of DNA sequences are provided to construct the dictionaries needed for the methods of current inventions. An example of such a collection is a collection of DNA sequences consisting of 6 nucleotides, wherein said DNA sequences differ from each other for at least 2 nucleotides, comprise at least 3 different nucleotides, do not comprise more than 2 consecutive identical nucleotides, and do not comprise any of AGAG, ACAC, ATAT, GAGA, GCGC, GTGT, CACA, CGCG, CTCT, TATA, TCTC or TGTG. More particularly a collection is provided consisting of 256 DNA sequences from which at least 50 DNA sequences are listed in Table 3.

In another aspect, a computer system for converting digital information into DNA molecules is provided, said computer system comprises one or more processors and is configured for performing the methods of the invention. In another aspect, a computer program for converting digital information into DNA molecules is provided, the computer program comprises instructions which, when the computer program product is executed by a computer, cause the computer to carry out the methods of the inventions.

In another aspect, a device for storing digital information is provided comprising a storage system for storing nucleotide sequences as synthesized in the methods of the invention.

In yet another aspect, a method of retrieving digital information from one or more of a plurality of synthesized DNA molecules is provided, wherein said synthesized DNA molecules encode a plurality of binary elements that encode the digital information, comprising:
amplifying (160) one or more of the plurality of synthesized DNA molecules;
sequencing (170) the amplified synthesized DNA molecules:
identifying nucleotides (180) storing digital information and information of the plurality of dictionaries used to convert binary elements into nucleotides;
converting (180) the nucleotides into the plurality of binary elements using the identified dictionaries; and
constructing (180) the digital information from the plurality of binary elements.

Said method optionally comprises a further step for correcting of errors. In one embodiment said DNA molecules are plasmids. It has been found that this method enables the DNA sequences to be read by any existing sequencing technology including nanopore technology using extremely small sequencing devices, such as but not limited to GridION, MinION, SmidgION. It is known that these sequencing devices have a high error rate. The method of this document can tolerate high amount of mutations. This is one of the advantages of the methods disclosed herein over the prior art methods. Because of the high error tolerance, production costs of the DNA storage technologies can be decreased, since cheaper but imperfect DNA synthesis methods could be used.

### DESCRIPTION OF THE DRAWINGS

FIG. 1 shows a workflow of the general encoding method.

FIG. 2 shows a workflow for decoding.

FIG. 3 shows an example of a photograph for encoding.

FIG. 4 shows an example of how bytes can be translated into DNA words using selected ones of a plurality of dictionaries.

FIG. 5 shows an example of the translation key or mask.

FIG. 6 shows an example of a 1779 nucleotide long DNA fragment encoding 345 bytes of information. The DNA fragment comprises 5 file units each consisting of 345 nucleotides each encoding 69 bytes, the mask code in quadruplicate, two copies of the fragment ID consisting of 16 nucleotides each and two copies of the file ID consisting of 3 nucleotides each.

FIG. 7 shows an example of a 982 nucleotide long DNA fragment encoding 148 bytes of information. Said fragment comprises 4 file data fragments, each consisting of 222 nucleotides (i.e. 37 words of 6 nucleotides), a file ID, fragment ID and mask ID. The file ID comprises 20 nucleotides and is present in duplicate, once at the start and once at the end of the DNA fragment. As such the file ID can be used for PCR primer annealing and thus for amplifying only one specific DNA fragment out of a plurality of DNA fragments. Also a fragment ID comprising 18 nucleotides is present in duplicate as well as a mask ID of 6 nucleotides in triplicate.

FIG. 8 shows an example of a 200 nucleotide long DNA fragment encoding 34 bytes of digital information. Said fragment comprises 1 file data fragment consisting of 136 nucleotides (i.e. 34 words of 4 nucleotides), a file ID, fragment ID (18 nucleotides) and mask ID (4 nucleotides). The file ID comprises 20 nucleotides and is present in duplicate, once at the start and once at the end of the DNA fragment.

FIG. 9 shows a workflow of the plasmid encoding method, whereby x can by any integer, e.g. x is 5.

FIG. 10 shows the number of reads needed per fragment (coverage) to obtain the encoded information using nanopore sequencing technology. A comparison is shown between the methods disclosed herein (light grey) and disclosed by Organick et al (dark grey).

FIG. 11 shows the retrieved text file that has been previously translated into DNA.

### DETAILED DESCRIPTION OF THE INVENTION

The invention will now be described on the basis of the drawings and with respect to particular embodiments. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The terms or definitions used herein are provided solely to aid in the understanding of the invention. Unless specifically defined herein, all terms used herein have the same meaning as they would to one skilled in the art of the present invention. Practitioners are particularly directed to Sambrook et al. (2012 Molecular Cloning: A Laboratory Manual, 4th ed., Cold Spring Harbor Press, Plainsview, N.Y.) and Ausubel et al. (2016 Current Protocols in Molecular Biology (Supplement 114), John Wiley & Sons, New York) for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The present application relates to a method for storage of digital information in DNA molecules. The method comprises an algorithm that is used to convert a file of information comprising digital data into artificial sequences of nucleotides, which can then be synthesised. This method was developed by the inventors to encode the binary information from the digital data into a sequence of nucleotides which can be synthesized and sequenced in an efficient and accurate manner without any further optimization of the digital or DNA code is needed. The core of the invention is that a set of optimized DNA elements (which will be referred to as “words”) are generated, that only said DNA elements or words are used in the translation process and that the translation key (i.e. which DNA element or word corresponds to which element of digital information) changes along the translation process. The method has been used to convert a plurality of different file extensions with a complex structure generated by the presence of a long series of similar digits. Current application additionally teaches the cloning of synthesized DNA fragments comprising digital data into plasmids, i.e. circular DNA molecules. Circular plasmids are extremely stable, as there are no ends from which degradation can easily occur. Plasmid are thus envisaged in the methods disclosed herein to improve long-term storage of DNA encoded digital information.

The method of current disclosure involves three tools: words, dictionaries and masks. Said terms will be explained in detail below.

Word, an Optimized DNA Element

A “word” as used herein refers to a precise sequence of a number of nucleotides (A C G T).

Because the nucleotide and its position are relevant parameters, it is possible to generate maximum 256 (i.e. 4⁴) different words of 4 nucleotides of length, 1024 (i.e. 4⁵) different words of 5 nucleotides, 4096 (i.e. 4⁶) different words of 6 nucleotides and so on. However, the length of the word and the amount of data it translates can be adapted. Given that there are 256 different combinations of 8 bits in a byte, the length of the word is preferably at least 4 nucleotides. In the Examples herein disclosed, Applicants used words of 4, 5 or 6 nucleotides to cover 1 byte (8 bits) of digital information. For storing digital data in oligonucleotides (<200 nucleotides) words of 4 nucleotides were used. For storing digital data in longer DNA fragments, words of 5 or 6 nucleotides were used. However, the skilled person in the art will appreciate that these examples are not limiting the invention and that both the length of the words and the amount of digital information can be adapted without deviating from the invention described herein. The term “word” will be interchangeably used herein with “DNA element”. In analogy, the term “digital element” will be used for a byte or any piece of digital information with an alternative length (e.g. 4, 5, 6, 7, . . . bits) which corresponds with a “word”.

In the example that the digital information is divided in bytes and that a 1 byte per word encoding is used, words of 5, 6 or more nucleotides as compared to 4 nucleotides have additional advantages. Indeed, having more words available then needed (256 possible combinations of 8 bits for a byte), allows a further selection of said words. For example, using only 256 words of 5 or 6 nucleotides out of the 1024 or 4096 available ones respectively, can increase the quality of the DNA synthesis and/or sequencing process and thus can improve the coding and decoding of digital data into DNA or vice versa. In one non-limiting aspect, the method specifies that each word used to encode the digital data should have at least two nucleotides different from any other of the words to be used. Although not essential to the invention, this approach facilitates error corrections. For example, in the case of a single mutation of the nucleotides in any one of the words, the altered (mutated) sequence cannot be confused with any of the other 255 words and hence the error can be easily detected and corrected. The method further specifies in a non-limiting aspect that words are selected by avoiding the DNA elements that would limit the efficiency of synthesis and sequencing of long DNA fragments. Non-limiting examples of words which are preferably removed from the selection of optimized words, are words that have more than 2 consecutive similar nucleotides (AAA, CCC, GGG, TTT) and words comprising one of the following patterns: AGAG, ACAC, ATAT, GAGA, GCGC, GTGT, CACA, CGCG, CTCT, TATA, TCTC, TGTG.

Dictionary, the Translation of a Word into a Digital Element

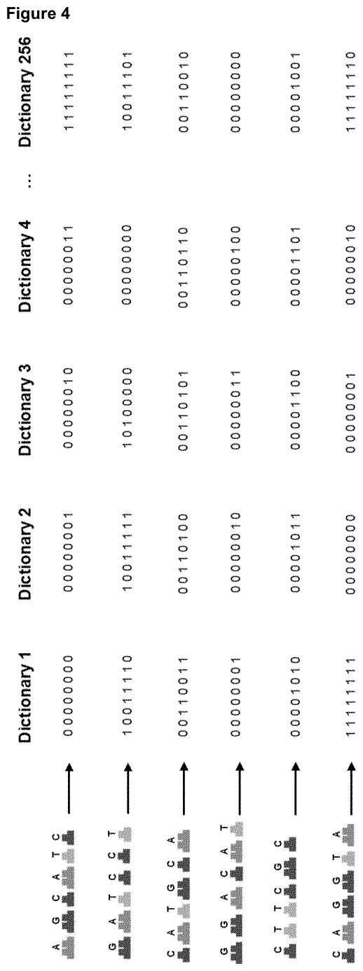

The group or set of “words” (e.g. 256 words to cover all 256 possible bytes) are used to form “dictionaries” (a type of hash table). The “dictionary” defines which word is connected to which digital element, e.g. byte. In a dictionary, each of the for example 256 words corresponds to a specific byte in the digital data. Different ones of the dictionaries can be generated by changing the order of the words in the dictionaries. A non-limiting example of this is shown in FIG. 4. It will be seen that in the first line the six-nucleotide word “AGCATC” can be translated in different sequences of 8 bits (or 1 byte). For example, in dictionary 1, “AGCATC” is translated into byte “00 00 00 00”, in dictionary 2 into “00 00 00 01”, in dictionary 256 into “11 11 11 11”, etc. It will be noted that this conversion is only exemplary and not limiting of the invention.

In total, 256 dictionaries can be used (and not just the five illustrated in FIG. 4). In different ones of the dictionaries the same word (e.g. group of six nucleotides) is related to a different byte of the digital data as will be seen in FIG. 4. Therefore, all the dictionaries are different from each other and none of the words have the same translation from the digital data between two different dictionaries. The number of possible dictionaries is thus reduced from 256! to 256. In case of a diverse digital code, a limited number of dictionaries may be sufficient to obtain a randomized DNA fragment which is efficiently synthesized and sequenced. In case of a repetitive digital sequence, it may be necessary to use a different dictionary for every byte that needs to be encoded.

Mask, the Dictionaries' Randomization Process

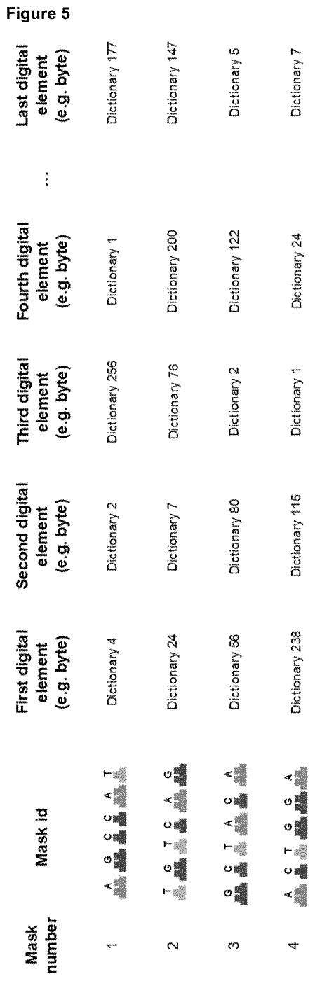

A dictionary allows the translation of a piece of the digital data (e.g. a byte) into a nucleotide sequence (i.e. word) as described above and be seen in FIG. 4. When the methods herein disclosed are used to translate a file of digital data into a highly diverse DNA fragment, the method constantly changes the dictionary used. Every element of digital information (e.g. 1 byte) that is encoded by a word is then translated using a different dictionary. The specific order of dictionaries that are used to translate a specific element of a digital file is determined by a translation key, herein referred to as “mask” and is shown in FIG. 5.

In the example in FIG. 5, using the first “mask”, the first byte of a digital file would be translated by the dictionary 4. The second byte by the dictionary 2, the third by dictionary 256, etc. The same first byte would be translated in the second mask not with the dictionary 4, but with a different dictionary 24, and in the third mask by dictionary 56, etc.

In one embodiment, the method uses 256 different masks to translate every digital file fragment. Hence, every file fragment can then be translated in at least 256 different DNA fragments. However, a skilled person in the art will appreciate that this is merely illustrative of the invention and the number of masks can be adapted and is not-limiting for current application. As a non-limiting example and only for the purpose of illustrating the herein disclosed reverse translation method and the technical effects thereof, the digital fragment consisting of 24 times the byte 0 is converted using mask 1 as shown in FIG. 5. The first byte would then be converted in GATCCT, the second in CAGGTA, the third in GGACAT and the last in AGCATC. A very repetitive digital fragment is thus converted in the diverse DNA fragment GATCCTCAGGTAGGACATAGCATC using mask 1 of which the information (i.e. AGCCAT) is then added to the DNA fragment.

From Digital Data to Storable DNA Fragment

In the end, the digital files that are translated into nucleotides have to be organized in DNA fragments. The invention as disclosed herein is compatible with all lengths of DNA fragments. For illustrative and non-limiting purposes, this is illustrated for 2 different fragment types in the Example section. The first type is “short oligonucleotides” (200 nucleotides or less), that are the cheapest and easiest to be produced. The second type is long DNA fragments (more than 300 nucleotides), that contain more information and redundancy in order to correct errors, but are more challenging to be synthetized and sequenced. Besides the nucleotide sequence harboring the digital information, additional information is needed. First of all, information is needed on which translation key or mask is used. This information is contained in the mask ID and identifies which randomization process has been selected in that specific fragment. As a non-limiting example, the mask ID can be 6 nucleotides long (as shown in FIG. 5). The mask ID can be shorter (e.g. 4 nucleotides) or longer. The longer a mask ID is, the more masks can be used and the more correction possibilities will be present when a mutation in a mask ID would occur. Second, a fragment ID is needed to identify which part of the file has been translated in that specific fragment. As a non-limiting example, the fragment ID can be 18 nucleotides long. Additionally, to obtain random access to a selected DNA fragment, every DNA fragment comprises a file specific sequence (e.g. 20 nucleotides) at the start and at the end, which can be used to anneal with DNA primers.

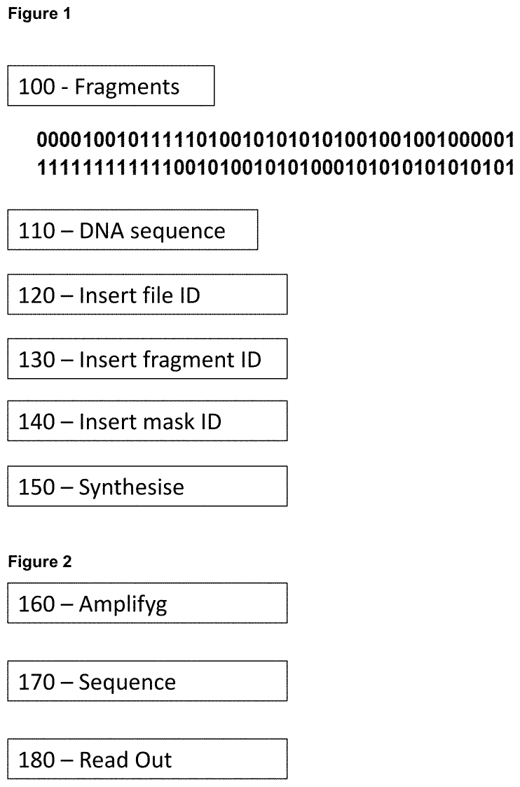

FIG. 1 shows a workflow of the method explained above. In a first step 100, the digital data is segmented into digital fragments. In one embodiment said fragments have a length of between 20 and 100 bytes, of between 50 and 200 bytes, of between 100 and 350 bytes or of between 200 and 1000 bytes. Every one of these digital fragments are then translated, in step 110, into a DNA fragment using the reverse translation principle herein disclosed and as illustrated above using FIGS. 4 and 5.

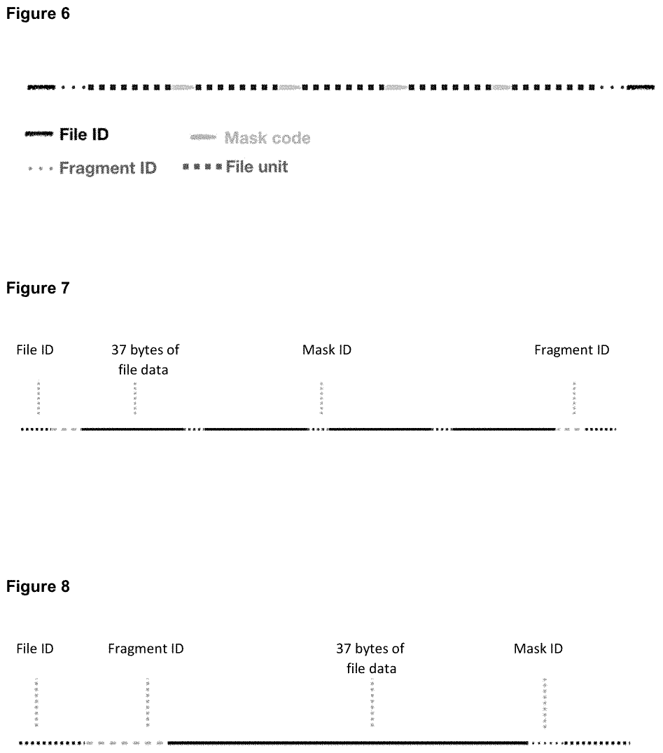

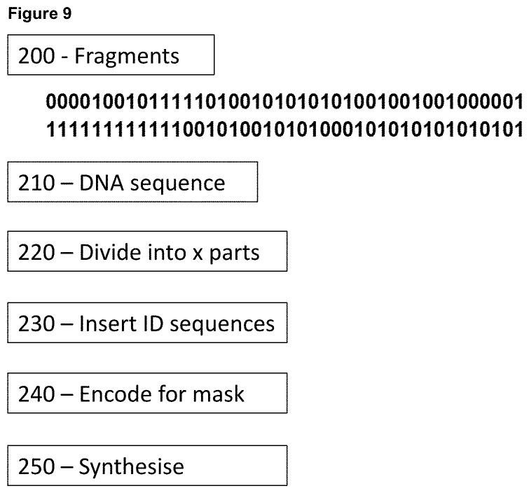

Non-limiting examples of how storable DNA fragments are constructed are shown in FIG. 6, 7 or 8, depending on the word length that is used and/or the kind of DNA structure (e.g. oligonucleotides or long DNA fragments). The example in FIG. 6 shows a fragment built by using words of 5 nucleotides of length for a total of 1779 nucleotides. The fragment was then cloned into plasmids. FIG. 7 shows a DNA fragment of 982 nucleotides built by using words of 6 nucleotides of length. FIG. 8 shows a fragment of 200 nucleotides built by using words of 4 nucleotides of length.

In case of multiple files being saved, every file has a specific file ID (120). The file ID is a DNA sequence, specific for each file. In some embodiments, the file ID can be used to anneal with specific primers that can be used to amplify only the selected file from a pool. Next, each DNA fragment is indexed by inserting the fragment ID (130). The fragment ID is necessary to order each fragment from the first to the last and thus retrieve all the data in the correct order. At this point, the binary information of each file fragment generated in (100) is translated by using a mask. Logically also the mask ID is therefore inserted into the DNA fragment (140). The resulting DNA fragment can be synthetized and stored (150).

Data Storage in Plasmids

As demonstrated in Example 1, the DNA fragments which are generated using the herein disclosed data storage method can be inserted into plasmids. Plasmids are extremely stable and resistant for degeneration and are therefore ideal storage molecules. A file plasmids library can be generated for example by using the commercially available library TwistKan plasmid as a vector.

FIG. 9 shows an exemplary workflow of the method using plasmids. In a first step 100, the digital data is segmented into fragments. In one embodiment said fragments have a length of between 20 and 100 bytes, of between 50 and 200 bytes, of between 100 and 350 bytes or of between 200 and 1000 bytes. In a most particular embodiment said fragments have a length of 345 bytes. Every one of these segments is then translated, in step 110, into a DNA sequence and subsequently cloned into the vector in step 150.

FIG. 6 illustrates the translation of the digital data into plasmids. As a non-limiting example, five inserts each corresponding to 69 bytes of digital information are shown in FIG. 6. It should be clear for the skilled one that the number of inserts can be adapted.

An exemplary plasmid is shown in FIG. 6. The two ID sequences inserted in steps 120 and 130 are the file ID and the fragment ID. The file ID consists of three nucleotides in this example and enables the storage of up to 64 different files inside a single library (i.e. 4³). It will be appreciated that the file ID of three nucleotides is a non-limiting example and in other embodiment of the methods any length of nucleotide sequences could be used as the file ID. The fragment ID consists of 16 nucleotides in this example and defines which part of the file is encoded in that specific plasmid. Similar to the file ID, the length of the fragment ID is not limiting the invention and in alternative embodiments any length of the nucleotide sequence can be used as the fragment ID. Between each part of the five inserts, there are four other ID codes inserted in step 140, which are 4 nucleotides each in length (in this example) and encode for the mask code. This inserted ID is basically defining the order of dictionaries that has been used to encode that specific file segment. It will be appreciated that any length of nucleotide sequence can be used as the mask code. This builds up altogether (in this non-limiting example) an encoded fragment with 1779 nucleotides (FIG. 6), in this example, which can then be synthesized in the step 150.

Additional to the storage and stability benefits of plasmids (as described above), the obtained plasmids can be inserted in microorganisms, for example bacteria. Instead of storing the synthesized DNA molecules, said microorganisms can be stored for example at −80° C. However, more interestingly said microorganisms can be used to amplify the plasmids comprising the digital information. Indeed, when the necessary molecular elements for replication are present in the backbone of said plasmids, said bacteria can easily amplify the plasmids to a very high level. Moreover, using plasmids to store digital information also allows a more advanced cataloging system combined with an additional tool to access particular files. This principle is explained in more detail by making use of a reading book comprising chapters as an example. The overall digital file, i.e. the reading book can be divided into digital fragments that for example represent the chapters of said book. Said digital fragments will be further divided in smaller digital fragments, for example first the pages of said chapters and further the sentences on said pages. All smallest digital fragments, for example all sentences on page x of chapter y of the reading book can then be stored in a plasmid with the same backbone comprising the same marker (e.g. a resistance gene for the antibiotic kanamycin). When only the information of page x of chapter y is to be retrieved, the bacterial collection is grown on medium with the corresponding antibiotic. In a next step the plasmids of the selected bacteria are isolated. Subsequently, very specific digital information (e.g. sentence 15 of page x of chapter y) can be amplified using the file specific sequences in the synthesized DNA fragment (see above) before a sequencing step is to be performed.

In a first aspect of the application as disclosed here, a method of storing information using DNA molecules is provided. Said method comprises the following steps:
(a) converting (100) a file of information into a plurality of fragments, wherein the plurality of fragments comprise or can be converted to a plurality of binary elements;
(b) converting (110) the plurality of binary elements into a plurality of nucleotides using selected ones of a plurality of dictionaries;
(c) constructing (120, 130, 140) a file unit comprising the plurality of nucleotides and an identification of the used ones of the plurality of dictionaries;
(d) synthesizing (150) a plurality of DNA molecules from the constructed file unit; and
(e) storing the plurality of synthesized DNA molecules.

In one embodiment, said information is digital information. In a more particular embodiment, said digital information is binary information. In one embodiment, the plurality of fragments from the step (a) are a plurality of digital fragments or fragments of digital information, more particularly of binary information. In another embodiment, said plurality of digital fragments or fragments of digital/binary information comprise a plurality of digital elements, wherein said digital elements are of or can be converted to binary elements consisting of 3, 4, 5, 6, 7 or 8 bits or of between 9 and 12 bits or of between 10 and 15 bits or of between 16 and 25 bits. In a particular embodiment, said plurality of binary elements are a plurality of bytes.

In one embodiment, said plurality of nucleotides are a plurality of DNA elements or “words” as defined by the definitions in current specification.

In one embodiment, said file unit additionally comprises an identification of which (digital) fragment from the file of information was converted to said plurality of nucleotides or alternatively said further comprises a fragment code indicating the position of the (digital) fragment in the file of (digital) information.

In a particular embodiment, said plurality of dictionaries comprise a plurality of DNA elements or “words” as defined by the definitions in current specification. In a more particular embodiment, said DNA elements consist of four, five or six nucleotides. In an even more particular embodiment, said DNA elements from said plurality of dictionaries differ from each other by at least two nucleotides. In one embodiment, said one of the plurality of dictionaries are used for converting (110) ones of the plurality of binary elements, more particularly of bytes. In a more particular embodiment, said plurality of binary elements from step (b) is converted into a plurality of nucleotides by different ones of the plurality of dictionaries. In even more particular embodiments, every binary element from said plurality of binary elements is converted by a different dictionary.

In particular embodiments, a step between step (d) and (e) is added, said step consists of combining two or more synthesized DNA molecules into a plasmid. Said combining can be done by molecular techniques of which the skilled one is familiar with, for example traditional molecular cloning. In alternative embodiments, a step between step (c) and (d) is added, said step consists of combining two or more constructed file units into a plasmid. Said combining can be done in silico after which the plasmid is synthesized in step (d). In both cases, in the final step of said extended methods, the obtained plasmid or plurality of plasmids are stored. In one further embodiment, at least two or at least three plasmids are generated and stored per digital fragment. In a particular embodiment, between 3 and 6, or between 4 and 8 or between 5 and 10 synthesized DNA molecules are combined into a plasmid. In more particular embodiments, said plasmids comprise a molecular marker. In even more particular embodiments, said plasmids comprise one or more antibiotic resistance genes such as “amp” for ampicillin, “strA” for streptomycin, etc.

Some of the methods steps disclosed above may be computer-implemented. The step of converting (110) the plurality of binary elements into a plurality of nucleotides using selected ones of a plurality of dictionaries is preferably computer-implemented. The step of constructing (120, 130, 140) a file unit comprising the plurality of nucleotides and an identification of the used ones of the plurality of dictionaries is preferably computer-implemented. The methods according to the first aspect may therefore be computer-implemented methods.

In a second aspect, the present invention provides a computer system for converting digital information into DNA, DNA molecules or nucleotides. The computer system comprises one or more processors. The computer system is configured for performing a method according the first aspect of the present invention.

In a third aspect, the present invention provides a computer program product for converting digital information into DNA, DNA molecules or nucleotides or for converting a plurality of binary elements into a plurality of nucleotides using selected ones of a plurality of dictionaries. The computer program product comprises instructions which, when the computer program product is executed by a computer, such as a computer system according to the second aspect of the present invention, cause the computer to carry out a method according to the first aspect of the present invention. In a fourth aspect, the present invention may furthermore provide a tangible non-transitory computer-readable data carrier comprising the computer program product. Also a device for storing digital information is provided, said device comprises a storage system for storing DNA molecules or nucleotide sequences synthesized according to the methods of the first aspect of the invention.

In a fifth aspect, a collection of DNA elements is provided, wherein said DNA elements consists of five nucleotides and wherein said DNA elements differ from each other for at least 2 nucleotides. In one embodiment, said collection comprises at least 50 DNA elements, at least 100 DNA elements, at least 150 DNA elements or at least 200 DNA elements. In a particular embodiment, said nucleotides are selected from the list consisting of A, T, G and C. In a most particular embodiment, said collection consists of 256 DNA elements as depicted in Table 1.

In a sixth aspect, a collection of DNA elements or DNA sequences consisting of six nucleotides is provided, wherein said DNA elements or sequences differ from each other for at least 2 nucleotides, comprise at least 3 different nucleotides, do not comprise more than 2 consecutive identical nucleotides, and do not comprise any of AGAG, ACAC, ATAT, GAGA, GCGC, GTGT, CACA, CGCG, CTCT, TATA, TCTC or TGTG. In one embodiment, said collection comprises at least 50 DNA elements, at least 100 DNA elements, at least 150 DNA elements or at least 200 DNA elements. More particularly, said at least 50 DNA elements, at least 100 DNA elements, at least 150 DNA elements or at least 200 DNA elements are listed in Table 2. In a particular embodiment, said nucleotides are selected from the list consisting of A, T, G and C. In a most particular embodiment, said collection consists of 256 DNA elements as depicted in Table 3.

In a seventh aspect, a method of retrieving digital information from one or more of a plurality of synthesized DNA molecules is provided, wherein said synthesized DNA molecules encode a plurality of binary elements that encode the digital information and wherein said plurality of binary elements was converted into said DNA molecules using selected or different ones of a plurality of dictionaries, said method comprises the following steps:
(a) amplifying (160) one or more of the plurality of synthesized DNA molecules;
(b) sequencing (170) the amplified synthesized DNA molecules:
(c) identifying nucleotides (180) storing digital information and storing information of said selected or different ones of the plurality of dictionaries;
(d) converting (180) the nucleotides into the plurality of binary elements using the identified dictionaries; and
(e) constructing (180) the digital information from the plurality of binary elements.

In one embodiment, said binary elements consist of 3, 4, 5, 6, 7 or 8 bits or of between 9 and 12 bits or of between 10 and 15 bits or of between 16 and 25 bits. In a particular embodiment, said plurality of binary elements are a plurality of bytes.

In one embodiment, said “nucleotides storing digital information” are a plurality of DNA elements or “words” as defined by the definitions in current specification and said “nucleotides storing dictionaries” comprises or consists of an identification of the used ones of the plurality of dictionaries as defined by the definitions in current specification.

In one embodiment, said method additionally comprises a step of identifying nucleotides storing information of which (digital) fragment from the file of (digital) information was converted to DNA molecules or alternatively said further comprises a step of identifying a fragment code indicating the position of the (digital) fragment in the file of (digital) information.

In another embodiment, said method further comprising a step of correcting of errors.

The skilled person in the art is aware of molecular techniques that can be used to amplify and sequence DNA molecules as referred to in step (a) and (b).

Some of the methods steps from the methods according to the seventh aspect of the invention may be computer-implemented. The step of identifying nucleotides (180) storing digital information and storing information of the dictionaries used to convert binary elements into nucleotides is preferably computer-implemented. The step of converting (180) the nucleotides into the plurality of binary elements using the identified dictionaries is preferably computer-implemented. The step of constructing (180) the digital information from the plurality of binary elements is preferably computer-implemented. The methods according to the seventh aspect may therefore be computer-implemented methods.

### EXAMPLES

In this application Applicants disclose a novel approach, i.e. a reverse translation approach to convert digital information into DNA and vice versa. The Examples below demonstrate how the method and modifications thereof can be reduced to practice.

### Example 1. DNA Fragments Made of Five Nucleotide Words

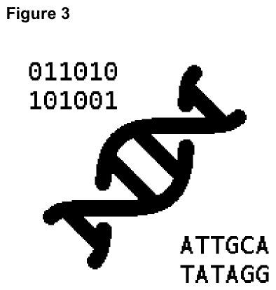

To test the method, two challenging files that are completely different from each other were used: the first page of the Divina Commedia poem by Dante and a black and white PNG image adapted for this purpose as shown in FIG. 3. The Divina Commedia TXT file (1380 bytes) is challenging because the file contains a lot of different bytes or characters. The image chosen (3450 bytes) is challenging for the opposite reason. It contains a series of 5832 times the bit 0. Such repetitive files cannot be translated either by the Goldman encoding bit-nucleotide standard way or by basic-encoding. The term “basic encoding” means using a code in which two bits are translated to one nucleotide, e.g. 00 is translated to A, 01 is translated to G, 01 is translated to C and 11 is translated to T. Similar to 1-bit to 1-nucleotide encoding, basic encoding is incompatible with current synthesis and sequencing methods as repetitions of 0 or 1 will create long series of repetitions such as oligopolymers.

It was decided to divide both files in fragments of 69 bytes and to use “words” (see detailed description) of 5 nucleotides. A collection of DNA elements was created consisting of 256 different 5 nucleotide-containing words wherein each word differed from each other with at least 2 nucleotides (Table 1).

As previously described, using the collection of 5 nucleotide words from Table 1, 256 different dictionaries were generated. Next and illustrated in FIG. 5, masks (or alternatively phrased: translation keys) were defined, describing which dictionaries will be used for the successive bytes that need to be translated into DNA elements or words. By doing so, all 345 bytes long digital fragments were translated into 5 DNA fragments of 345 nucleotides each and the mask ID consisting of 4 nucleotides determining which combination of dictionaries was used was added. In total, 8 plasmids for the Divina commedia and 20 for the picture of FIG. 3 have been synthetized. Additionally, in order to have more cloning flexibility later on, the plasmids have been selected to not contain both EcoRI and BamHI restriction sites (that are, respectively, GTTAAC and GGATCC). The list of all the fragments and the masks we used can be found in Table 2.

TABLE 1
Set of 256 different 5-nucleotide long DNA
sequences (herein referred to as “words”)

| | | | | | | | |
|---|---|---|---|---|---|---|---|
| TCAAG | TAAAT | CCAAA | CAAAC | GCAAT | GAAAG | ACAAC | AAAAA |
| TCAGA | TAAGC | CCAGG | CAAGT | GCAGC | GAAGA | ACAGT | AAAGG |
| TCACT | TAACG | CCACC | CAACA | GCACG | GAACT | ACACA | AAACC |
| TCATC | TAATA | CCATT | CAATG | GCATA | GAATC | ACATG | AAATT |
| TCGAA | TAGAC | CCGAG | CAGAT | GCGAC | GAGAA | ACGAT | AAGAG |
| TCGGG | TAGGT | CCGGA | CAGGC | GCGGT | GAGGG | ACGGC | AAGGA |
| TCGCC | TAGCA | CCGCT | CAGCG | GCGCA | GAGCC | ACGCG | AAGCT |
| TCGTT | TAGTG | CCGTC | CAGTA | GCGTG | GAGTT | ACGTA | AAGTC |
| TCCAT | TACAG | CCCAC | CACAA | GCCAG | GACAT | ACCAA | AACAC |
| TCCGC | TACGA | CCCGT | CACGG | GCCGA | GACGC | ACCGG | AACGT |
| TCCCG | TACCT | CCCCA | CACCC | GCCCT | GACCG | ACCCC | AACCA |
| TCCTA | TACTC | CCCTG | CACTT | GCCTC | GACTA | ACCTT | AACTG |
| TCTAC | TATAA | CCTAT | CATAG | GCTAA | GATAC | ACTAG | AATAT |
| TCTGT | TATGG | CCTGC | CATGA | GCTGG | GATGT | ACTGA | AATGC |
| TCTCA | TATCC | CCTCG | CATCT | GCTCC | GATCA | ACTCT | AATCG |
| TCTTG | TATTT | CCTTA | CATTC | GCTTT | GATTG | ACTTC | AATTA |
| TTAAA | TGAAC | CTAAG | CGAAT | GTAAC | GGAAA | ATAAT | AGAAG |
| TTAGG | TGAGT | CTAGA | CGAGC | GTAGT | GGAGG | ATAGC | AGAGA |
| TTACC | TGACA | CTACT | CGACG | GTACA | GGACC | ATACG | AGACT |
| TTATT | TGATG | CTATC | CGATA | GTATG | GGATT | ATATA | AGATC |
| TTGAG | TGGAT | CTGAA | CGGAC | GTGAT | GGGAG | ATGAC | AGGAA |
| TTGGA | TGGGC | CTGGG | CGGGT | GTGGC | GGGGA | ATGGT | AGGGG |
| TTGCT | TGGCG | CTGCC | CGGCA | GTGCG | GGGCT | ATGCA | AGGCC |
| TTGTC | TGGTA | CTGTT | CGGTG | GTGTA | GGGTC | ATGTG | AGGTT |
| TTCAC | TGCAA | CTCAT | CGCAG | GTCAA | GGCAC | ATCAG | AGCAT |
| TTCGT | TGCGG | CTCGC | CGCGA | GTCGG | GGCGT | ATCGA | AGCGC |
| TTCCA | TGCCC | CTCCG | CGCCT | GTCCC | GGCCA | ATCCT | AGCCG |
| TTCTG | TGCTT | CTCTA | CGCTC | GTCTT | GGCTG | ATCTC | AGCTA |
| TTTAT | TGTAG | CTTAC | CGTAA | GTTAG | GGTAT | ATTAA | AGTAC |
| TTTGC | TGTGA | CTTGT | CGTGG | GTTGA | GGTGC | ATTGG | AGTGT |
| TTTCG | TGTCT | CTTCA | CGTCC | GTTCT | GGTCG | ATTCC | AGTCA |
| TTTTA | TGTTC | CTTTG | CGTTT | GTTTC | GGTTA | ATTTT | AGTTG |

All obtained DNA fragments were found to be synthesizable according to three different types of DNA synthesis commercial companies (Twist Bioscience, IDT and SGI-DNA). The synthesis was done into logical duplicate, so that there was redundancy to minimize the effects of any errors. An advantage of this kind of encoding methodology is that we can synthesize several different logical copies of any files.

TABLE 2
All the masks used and the plasmids synthetized for encoding
the first page of Divina Commedia and the image in FIG. 3.

| Mask | Plasmid name |
|---|---|
| 2 | Dante_A1 |
| 3 | Dante_A2 |
| 2 | Dante_B1 |
| 4 | Dante_B2 |
| 2 | Dante_C1 |
| 5 | Dante_C2 |
| 1 | Dante_D1 |
| 2 | Dante_D2 |
| 5 | DNA_A1 |
| 6 | DNA_A2 |
| 253 | DNA_B1 |
| 254 | DNA_B2 |
| 3 | DNA_C1 |
| 4 | DNA_C2 |
| 3 | DNA_D1 |
| 5 | DNA_D2 |
| 3 | DNA_E1 |
| 6 | DNA_E2 |
| 10 | DNA_F1 |
| 4 | DNA_F2 |
| 2 | DNA_G1 |
| 10 | DNA_G2 |
| 2 | DNA_H1 |
| 4 | DNA_H2 |
| 1 | DNA_I1 |
| 3 | DNA_I2 |
| 3 | DNA_J1 |
| 8 | DNA_J2 |

In addition to these wet biology experiments, the method was tested in silico with 3 other different files: a PDF, a colored image and a mp3 audio file. All of the additionally tested files resulted in synthesizable sequences for all of the three different commercial companies.

We reasoned that for storage purposes it might be advantageous to clone the obtained DNA fragments in plasmids (FIG. 9). Plasmids are known to be more stable and degradation resistant compared to linear DNA molecules. Therefore, plasmids were generated comprising 5 inserts of 345 nucleotide long DNA fragments each (step 220 in FIG. 9), together with their corresponding file ID, fragment ID and mask ID (steps 230 and 240). It should however be clear that cloning into plasmids is optional and does not limit the methods as herein disclosed.

After the files have been synthesized (step 250), and optionally cloned in plasmids, they were sequenced in step 160 in order to retrieve the information as is shown in FIG. 2. The method of retrieving digital information from the synthesized DNA molecules comprises amplifying the DNA sequence in step 160, sequencing the molecule in step 170 and reading out the results in step 180. The step 180 can include error detection and correction. Briefly, the DNA sequences from step 170 are checked in order to confirm that every sequence contains valid IDs and “words”. In case an invalid DNA sequence is found, it can be corrected or, when not possible, just excluded.

For both the Divina Commedia file and the PNG image, Sanger sequencing was successfully performed using extremely low dilutions (<0.1 pg of DNA) as a template for amplifying the DNA sequence in step 160. We have found no mutations or plasmid dropout. Additionally, sequencing was simulated using NanoSim simulator (a scalable read simulator that captures the technology-specific features of ONT data) and pIRS (profile based Illumina pair-end Reads Simulator) to check whether the files are compatible with Illumina NGS and Gridion Oxford Nanopore sequencing technologies. It was found that after simulating the sequencing there were no errors present and the method was able to retrieve all of the information in the files in step 180 with both sequencing methods.

One limit to the data-into-DNA storage is the risks of mutations, dropout and errors that can be introduced by synthesis, amplification, sequencing and aging. Particularly the amount of said DNA alterations will be crucial.

In order to challenge the reverse translation method, a different amount and type of mutations were introduced in silico and the method was then tested to see if it was able to retrieve the information in the files. These simulations revealed that is possible to retrieve the information from the files, 10 times out of 10, after introducing one random mutation (insertion, deletion or substitution) in 100% of our plasmids. The number of mutations was also increased up to 1 mutation every 100 base pairs inside our plasmids. The method was able to retrieve the file 10 times out of 10 random trials.

### Example 2. Long DNA Fragments Made of Six Nucleotide Words

Next, the use of a different word length (i.e. 6 nucleotides) was demonstrated. The advantage of 6 nucleotide words is that the method can be even further optimized for the synthesis of long DNA fragments and for sequencing technologies such as Oxford Nanopore Technology, which has rather high error rates per reads.

From the 4096 possible combinations of 6 nucleotides (4⁶), a set of 256 words was selected (Table 3). Each word of 6 nucleotides we have generated went through several optimization steps. It was found that said words had to fulfill the following criteria:
(i) words should not comprise more than 2 consecutive similar nucleotides (AAA, CCC, GGG, TTT) per word;
(ii) every word must comprise at least 3 different nucleotides;
(iii) the following patterns, inside a word, are forbidden: AGAG, ACAC, ATAT, GAGA, GCGC, GTGT, CACA, CGCG, CTCT, TATA, TCTC or TGTG;
(iv) every word has to comprise at least 2 nucleotides difference with other words or all words should differ from each other for at least 2 nucleotides.

Among all the 688 valid words that were created with those parameters, 256 words were selected for creating dictionaries. The selection is shown in Table 3.

TABLE 3
Set of 256 different 6-nucleotide long DNA
sequences (herein referred to as “words”)

| | | | | | | | |
|---|---|---|---|---|---|---|---|
| TCGCAT | GTTCGT | GCTTAC | CTTATC | CCTGAT | ATTCCT | AGCCTG | AACCAG |
| TCGTCA | GTTGCT | GGAATC | CTTCCG | CCTGGC | ATTGAC | AGCGGA | AACCGA |
| TCTAAT | GTTGTC | GGACAT | CTTCGC | CCTTAG | ATTGCA | AGCGTC | AACGCA |
| TCTAGC | TAAGGC | GGACGC | GAACGT | CGAATT | ATTGGT | AGCTTA | AACGGT |
| TCTGCA | TAATGA | GGAGTT | GAACTG | CGACTG | CAAGAC | AGGATA | AAGCAC |
| TCTTAA | TACAGG | GGATAC | GAAGCT | CGATCG | CAAGGT | AGGTCC | AAGCCA |
| TCTTGG | TACCAC | GGATCA | GAAGTC | CGATGC | CACCAT | AGGTGG | AAGTGC |
| TGAAGC | TACCGT | GGATGT | GAATCG | CGCCAC | CACCGC | AGTACT | AATAGT |
| TGACAG | TACGAG | GGCAAT | GAATTA | CGCTGA | CACGAA | AGTAGA | AATCGG |
| TGACCT | TACGTC | GGCAGC | GACATG | CGCTTC | CACGCC | AGTCAT | AATGGC |
| TGACGA | TACTGC | GGCGTG | GACGTA | CGGACT | CACGTT | AGTTAC | AATTCT |
| TGAGCA | TAGACG | GGCTAA | GACTTC | CGGTCA | CACTCA | ATAACA | ACAATA |
| TGAGGT | TAGATA | GGCTCC | GAGCAT | CGGTTG | CAGCAA | ATAAGG | ACAGGT |
| TGAGTG | TAGCCT | GGTAAC | GAGCTA | CGTAAT | CAGCTT | ATACAA | ACATCC |
| TGCACT | TAGCTC | GGTAGT | GATAAT | CGTACG | CAGGAT | ATACTG | ACCACT |
| TGCATC | TAGGTG | GGTATG | GATCAG | CGTCAG | CAGGTA | ATCATG | ACCGAA |
| TGCGAA | TAGTCC | GGTCTT | GATGCA | CGTTAA | CAGTTC | ATCCGG | ACCGCC |
| TGCTGT | TAGTGG | GGTGGC | GATGGT | CGTTGG | CATACC | ATCCTT | ACCGTT |
| TGCTTG | TATCTA | GGTTAG | GCAAGT | CTAACC | CATAGG | ATCGAT | ACCTGT |
| TGGACA | TATGAA | GTCAAG | GCACGG | CTACCA | CATGGA | ATCGGC | ACGCTT |
| TGGCGG | TATGCC | GTCACT | GCATTC | CTAGAT | CATTAT | ATCGTA | ACGGCG |
| TGGTCT | TATTAC | GTCCTA | GCCAGG | CTAGCG | CATTCG | ATCTAG | ACGTGA |
| TGTCCA | TATTGT | GTCGAA | GCCATT | CTAGGC | CCAATC | ATGAAG | ACGTTC |
| TGTTGC | TCAAGG | GTCGTT | GCCGAG | CTATCT | CCACCG | ATGATC | ACTAGG |
| TTAGAA | TCACGT | GTCTAC | GCCGGA | CTATGA | CCAGAA | ATGCAT | ACTCCA |
| TTAGTT | TCACTG | GTGAAC | GCCTGC | CTCATT | CCATAC | ATGCCG | ACTCGT |
| TTCAAT | TCAGCT | GTGACA | GCGACG | CTCGGA | CCATGT | ATGGAA | ACTGGA |
| TTCGGT | TCATGC | GTGATG | GCGGAC | CTGAAT | CCGATT | ATGGCC | ACTGTC |
| TTCTAA | TCATTA | GTGCGG | GCTAGA | CTGACG | CCGCTG | ATGGTT | AGACCA |
| TTGCTG | TCCATG | GTGGAT | GCTCGC | CTGGCA | CCGGCT | ATGTAC | AGACTT |
| TTGGCT | TCCGGC | GTGGCG | GCTGCC | CTGTAA | CCGTGC | ATTAGC | AGATGA |
| TTGTCG | TCGAAG | GTTAGG | GCTGTT | CTTAAG | CCTCAA | ATTCAG | AGCAAC |

By using the herein disclosed reverse translation method and a plurality of dictionaries consisting of 256 optimized words of 6 nucleotides, it was investigated whether digital files could be translated into long DNA fragments (illustrated in FIG. 7). Each fragment is 982 nucleotides of length and encoded 148 bytes. Each byte has been converted into DNA sequences of 6 nucleotides each (Table 3). Two file ID sequences of 20 bps have been included at each extremity of the fragment, functioning as annealing sequences for a forward and a reverse primer. Moreover, 2 fragment IDs of 18 base pairs each (step 130) and 3 mask IDs of 6 base pairs each (step 140) have been included in the fragment. The resulting fragments of 982 nucleotides can be ordered as gBlocks from IDT, that are high quality (low mutations rate and high purification) DNA fragments.

The quality check algorithms of three of the most important commercial synthesis companies (IDT, SGI-DNA and Twist Bioscience) resulted into a 100% synthesis efficiency in silico for a 200 Mb txt file.

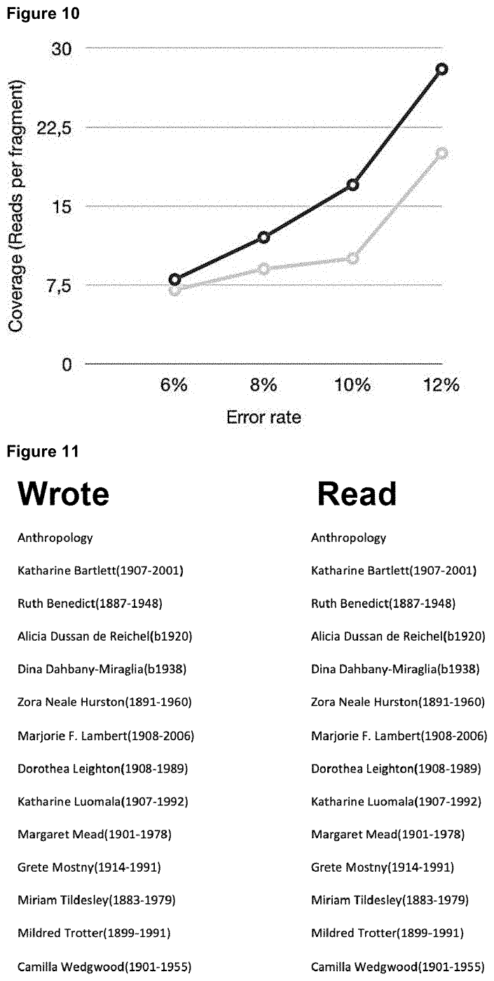

Next, the error-correction efficiency of our method was tested by simulating an Oxford Nanopore Technology (ONT) sequencing on a 200 Mb txt file translated into DNA. We stepwise increased the number or errors per reads, from 6% to 12%, distributed in 30% deletions, 30% insertions and 40% substitutions (that is the frequency that occurs in ONT sequencing) and simulated the coverage needed in order to retrieve the file. We compared our results to an analogous simulation made by Organick et al. (2018 Nat Biotech 36: 242-249). Surprisingly, current approach needs a lower coverage compared to Organick et al. (FIG. 10).

After that, the synthesis efficiency was tested with a real experiment in vitro. We translated a txt file of 7000 bytes, revealing a list of the most important female scientists of the 20^{th} century as retrieved from Wikipedia (listoffemalescientists20cen.zip), and a black and white picture (of 11900 bytes) of Rosalind Franklin. Because of copyright reason, the picture of Rosalind Franklin is not reproduced herein. In total, we encoded 27972 bytes, including 18900 bytes of data and 9072 bytes of Reed-Solomon redundancy, which is an error correcting code for retrieving corrupt data or errors in specific sequences. The file has been translated as previously described (illustrated in FIG. 7), and in total 189 DNA fragments (70 for the “txt” and 119 for the “picture” files) of 982 nucleotides each were ordered as gBlocks from IDT. A final density of 0.81 bits per nucleotide was achieved.

Subsequently, all fragments were sequenced using MiniON from ONT and error rates were calculated. Interestingly, because only optimized structures that are easy to be read are used, an error rate of about 10% per read was obtained. Other works (e.g. Yadzi et al. or Organick et al.) normally have about 20% more errors. Additionally, by using only 700 reads of the 70 fragments encoding the “txt file” (i.e. 10 randomly selected reads per fragment by reading the fragment ID), we were able to retrieve the file without any error (FIG. 11). Other works (e.g. Yadzi et al. or Organick et al.) normally need about 4 times more coverage (reads per fragment) compared to the herein disclosed methods.

It is clear for the skilled person that the approach explained in Example 2 is compatible with storing DNA fragments into plasmids as well.

### Example 3. Oligonucleotides Made of 4 Nucleotide Words

Because synthesis costs increase by increasing fragment length, most data-into-DNA storage approaches make use of oligonucleotides, i.e. DNA fragment of less than 100 nucleotides. Here, it is demonstrated that the current invention is fully compatible with oligonucleotides as well. For this approach we decided to use words of 4 nucleotides.

In case a digital information fragment will be encoded byte per byte, dictionaries will be generated for the conversion of the 256 different bytes. When words of 4 nucleotides will be used (see Table 4 for a collection of 256 different words of 4 nucleotides), it will therefore not be possible to make a selection from the 256 possible words. However, it is still possible to create oligos that do not contain any difficult to synthesize or sequence structure (e.g. AAAA) by selecting masks from a pool of different ones.

TABLE 4
Set of 256 different 4-nucleotide long DNA
sequences (herein referred to as “words”)

| | | | | | | | |
|---|---|---|---|---|---|---|---|
| TGAA | TAAA | GGAA | GAAA | CGAA | CAAA | AGAA | AAAA |
| TGAC | TAAC | GGAC | GAAC | CGAC | CAAC | AGAC | AAAC |
| TGAG | TAAG | GGAG | GAAG | CGAG | CAAG | AGAG | AAAG |
| TGAT | TAAT | GGAT | GAAT | CGAT | CAAT | AGAT | AAAT |
| TGCA | TACA | GGCA | GACA | CGCA | CACA | AGCA | AACA |
| TGCC | TACC | GGCC | GACC | CGCC | CACC | AGCC | AACC |
| TGCG | TACG | GGCG | GACG | CGCG | CACG | AGCG | AACG |
| TGCT | TACT | GGCT | GACT | CGCT | CACT | AGCT | AACT |
| TGGA | TAGA | GGGA | GAGA | CGGA | CAGA | AGGA | AAGA |
| TGGC | TAGC | GGGC | GAGC | CGGC | CAGC | AGGC | AAGC |
| TGGG | TAGG | GGGG | GAGG | CGGG | CAGG | AGGG | AAGG |
| TGGT | TAGT | GGGT | GAGT | CGGT | CAGT | AGGT | AAGT |
| TGTA | TATA | GGTA | GATA | CGTA | CATA | AGTA | AATA |
| TGTC | TATC | GGTC | GATC | CGTC | CATC | AGTC | AATC |
| TGTG | TATG | GGTG | GATG | CGTG | CATG | AGTG | AATG |
| TGTT | TATT | GGTT | GATT | CGTT | CATT | AGTT | AATT |
| TTAA | TCAA | GTAA | GCAA | CTAA | CCAA | ATAA | ACAA |
| TTAC | TCAC | GTAC | GCAC | CTAC | CCAC | ATAC | ACAC |
| TTAG | TCAG | GTAG | GCAG | CTAG | CCAG | ATAG | ACAG |
| TTAT | TCAT | GTAT | GCAT | CTAT | CCAT | ATAT | ACAT |
| TTCA | TCCA | GTCA | GCCA | CTCA | CCCA | ATCA | ACCA |
| TTCC | TCCC | GTCC | GCCC | CTCC | CCCC | ATCC | ACCC |
| TTCG | TCCG | GTCG | GCCG | CTCG | CCCG | ATCG | ACCG |
| TTCT | TCCT | GTCT | GCCT | CTCT | CCCT | ATCT | ACCT |
| TTGA | TCGA | GTGA | GCGA | CTGA | CCGA | ATGA | ACGA |
| TTGC | TCGC | GTGC | GCGC | CTGC | CCGC | ATGC | ACGC |
| TTGG | TCGG | GTGG | GCGG | CTGG | CCGG | ATGG | ACGG |
| TTGT | TCGT | GTGT | GCGT | CTGT | CCGT | ATGT | ACGT |
| TTTA | TCTA | GTTA | GCTA | CTTA | CCTA | ATTA | ACTA |
| TTTC | TCTC | GTTC | GCTC | CTTC | CCTC | ATTC | ACTC |
| TTTG | TCTG | GTTG | GCTG | CTTG | CCTG | ATTG | ACTG |
| TTTT | TCTT | GTTT | GCTT | CTTT | CCTT | ATTT | ACTT |

The structure used for the oligo is summarized in FIG. 8. Two file ID sequences of 20 bps have been included at each extremity of the fragment, functioning as annealing sequences for a forward and a reverse primer. After the forward primer sequence, a fragment IDs of 18 base pairs (step 130) has been added. The mask IDs of 6 base pairs each (step 140) have been added before the reverse primer sequence. In the middle, 34 “words” of 4 nucleotides each translate 34 bytes of information. In total, the oligo nucleotides are 200 bps of length. Of notice, in this case, all the 688 words of 6 nucleotides previously generated have been used to generate the mask ID. In this way, more oligo combinations can be generated and the selection can be stricter.

As an example of how the data-to-DNA translation works and how nucleic acids can be constructed, the translation of the following sentence of 68 bits/characters: “This txt file is our first test to store digital information in DNA.” is illustrated below. Said sentence is translated into the following 2 exemplary oligonucleotides, each consisting of a file ID (forward and reverse), a fragment ID, 34 bytes of data, and a mask ID.

First oligo:
AAGGCAAGTTGTTACCAGCATTATTGTCGCCGACGGCGATGGCACCGATT
TCCCGTAGCATCGATGGCAGTCCGTCTTTGGTTACCTCCGCATCCGCAAC
ATCTGGCAGTACAATTTACAATGCGTGTTAAGGGTCTATCATGGCAAAGT
AGTCTACTCACAGTCGACCTCGGAAAGTCGTTGGTTTGATTACGGTCGC
A
Forward Primer File ID (File 1):
AAGGCAAGTTGTTACCAGCA
Fragment ID (Fragment 1):
TTATTGTCGCCGACGGCG
Data (34 bytes):
ATGGCACCGATTTCCCGTAGCATCGATGGCAGTCCGTCTTTGGTTACCTC
CGCATCCGCAACATCTGGCAGTACAATTTACAATGCGTGTTAAGGGTCTA
TCATGGCAAAGTAGTCTACTCACAGTCGACCTCGGA
Mask ID (23):
AAGTCG
Reverse Primer File ID (File1):
TTGGTTTGATTACGGTCGCA
Second oligo:
AAGGCAAGTTGTTACCAGCATGGAGTTGCATCATAACATGAGCCTCCGGC
TATCTTGCAGGTATGGATAGATGGTCCGGTATACCGTCCAAGACTATGGC
TCGGCGTCATTGGTCTGGGAAGCACCTAGTGTTGTAGCAGGGACTATGCG
GCATCGCTACTCCCTACGTAAGTACGTGGTTTGGTTTGATTACGGTCGC
A
Forward Primer File ID (File 1):
AAGGCAAGTTGTTACCAGCA
Fragment ID (Fragment 2):
TGGAGTTGCATCATAACA
Data (34 bytes):
TGAGCCTCCGGCTATCTTGCAGGTATGGATAGATGGTCCGGTATACCGTC
CAAGACTATGGCTCGGCGTCATTGGTCTGGGAAGCACCTAGTGTTGTAGC
AGGGACTATGCGGCATCGCTACTCCCTACGTAAGTAC
Mask ID (294):
GTGGTT
Reverse Primer File ID (File1):
TGGTTTGATTACGGTCGCA

1. A method of storing digital information using DNA molecules, the method comprising:
(a) converting a file of digital information into a plurality of fragments, wherein the plurality of fragments comprise or are converted to a plurality of binary elements;
(b) converting the plurality of binary elements into a plurality of nucleotides utilizing dictionaries selected from a plurality of dictionaries, wherein a dictionary is individually selected from the plurality of dictionaries for the conversion of each binary element into a nucleotide;
(c) constructing a file unit comprising the plurality of nucleotides and an identification of the selected dictionaries;
(d) synthesizing a plurality of DNA molecules from the constructed file unit; and
(e) storing the plurality of synthesized DNA molecules.

2. The method according to claim 1, wherein each of the plurality of dictionaries comprises a plurality of members, and wherein the members consist of four, five, or six nucleotides.

3. The method according to claim 2, wherein the each of the members of the dictionaries consisting of five or six nucleotides differ from each other by at least two nucleotides.

4. The method according to claim 1, wherein at least two different dictionaries are selected.

5. The method according to claim 1, wherein the DNA molecules are plasmids.

6. The method according to claim 5, wherein at least three plasmids are synthesized and stored per fragment.

7. The method according to claim 1, wherein the file unit further comprises a fragment code indicating the position of the plurality of fragments in the file of digital information.

8. A computer system for converting digital information into DNA molecules, the computing system comprising one or more processors, the computing system configured for performing the method according to claim 1.

9. (canceled)

10. (canceled)

11. A method of retrieving digital information from one or more of a plurality of synthesized DNA molecules, wherein the synthesized DNA molecules encode a plurality of binary elements that encode the digital information, the method comprising:
(a) amplifying one or more of the plurality of synthesized DNA molecules;
(b) sequencing the amplified synthesized DNA molecules:
(c) identifying nucleotides storing digital information and identifying, from the sequencing, the dictionaries used to convert binary elements into nucleotides;
(d) converting the nucleotides into the plurality of binary elements using the identified dictionaries; and
(e) constructing the digital information from the plurality of binary elements.

12. The method according to claim 11, further comprising a step of correcting of errors.

13. The method according to claim 11, wherein said DNA molecules are plasmids.

14. The method according to claim 3, wherein each of the members:
consists of 6 nucleotides,
comprises at least 3 different nucleotides,
does not comprise more than 2 consecutive identical nucleotides, and
does not comprise any of AGAG, ACAC, ATAT, GAGA, GCGC, GTGT, CACA, CGCG, CTCT, TATA, TCTC or TGTG.

15. The method according to claim 14, wherein members consist of 256 DNA sequences from which at least 50 DNA sequences are listed in Table 3.


SEQUENCE LISTING (10 sequences)

SEQ ID NO: 1 - DNA, 24 bp, Artificial Sequence
  diverse DNA fragment
gatcctcaggtaggacatagcatc

SEQ ID NO: 2 - DNA, 200 bp, Artificial Sequence
  First oligo
aaggcaagttgttaccagcattattgtcgccgacggcgatggcaccgatttcccgtagcatcgatggcagtccgtctttggttacctccgcatccgcaacatctggcagtacaatttacaatgcgtgttaagggtctatcatggcaaagtagtctactcacagtcgacctcggaaagtcgttggtttgattacggtcgca

SEQ ID NO: 3 - DNA, 20 bp, Artificial Sequence
  Primer
aaggcaagttgttaccagca

SEQ ID NO: 4 - DNA, 18 bp, Artificial Sequence
  Fragment ID
ttattgtcgccgacggcg

SEQ ID NO: 5 - DNA, 136 bp, Artificial Sequence
  Data
atggcaccgatttcccgtagcatcgatggcagtccgtctttggttacctccgcatccgcaacatctggcagtacaatttacaatgcgtgttaagggtctatcatggcaaagtagtctactcacagtcgacctcgga

SEQ ID NO: 6 - DNA, 20 bp, Artificial Sequence
  Primer
ttggtttgattacggtcgca

SEQ ID NO: 7 - DNA, 200 bp, Artificial Sequence
  Oligo
aaggcaagttgttaccagcatggagttgcatcataacatgagcctccggctatcttgcaggtatggatagatggtccggtataccgtccaagactatggctcggcgtcattggtctgggaagcacctagtgttgtagcagggactatgcggcatcgctactccctacgtaagtacgtggtttggtttgattacggtcgca

SEQ ID NO: 8 - DNA, 18 bp, Artificial Sequence
  Fragment ID
tggagttgcatcataaca

SEQ ID NO: 9 - DNA, 137 bp, Artificial Sequence
  Data
tgagcctccggctatcttgcaggtatggatagatggtccggtataccgtccaagactatggctcggcgtcattggtctgggaagcacctagtgttgtagcagggactatgcggcatcgctactccctacgtaagtac

SEQ ID NO: 10 - DNA, 19 bp, Artificial Sequence
  Primer
tggtttgattacggtcgca